(19) United States
(12) Patent Application Publication
(10) Pub. No.: US 2021/0212946 A1
(43) Pub. Date: Jul. 15, 2021

(54) SOLID SELF-EMULSIFYING CANNABINOID COMPOSITIONS

(71) Applicant: ICDPHARMA LTD., Carme-Yosef (IL)

(72) Inventor: Doron FRIEDMAN, Carme-Yosef (IL)

(21) Appl. No.: 16/959,904

(22) PCT Filed: Jan. 2, 2019
(86) PCT No.: PCT/IL2019/050009

§ 371 Date: Jul. 22, 2020

Related U.S. Application Data
(60) Provisional application No. 62/613,202, filed on Jan. 3, 2018.

(51) Int. Cl. A61K 9/107; A61K 47/44; A61K 36/185; A61K 31/05; A61K 31/352; A61K 47/14; A61K 9/16; A61K 47/02; A61K 47/32; A61K 47/38; A61K 9/28; A61K 9/48; A61K 9/00

(52) U.S. Cl. CPC A61K 9/1075; A61K 9/0056; A61K 36/185; A61K 31/05; A61K 31/352; A61K 47/14; A61K 9/16; A61K 47/02; A61K 47/32; A61K 47/38; A61K 9/2846; A61K 9/2853; A61K 9/4816; A61K 9/006; A61K 47/44

(57) ABSTRACT

The present invention provides a solid self-emulsifying cannabinoid composition, comprising at least one cannabinoid or a mixture thereof, at least one essential oil or at least one terpene or a mixture thereof, at least two emulsifiers, and at least one adsorbing powder, wherein the cannabinoid(s) are essentially solubilized in the mixture of the terpene(s), essential oil(s) and at least two emulsifiers, wherein the resulting mixture is adsorbed onto the adsorbing powder(s) affording a solid self-emulsifying composition. The invention also provides methods for using the composition of the invention and a kit containing said composition.

### CROSS REFERENCE TO RELATED APPLICATIONS

This application claims priority to U.S. Provisional Patent Application Ser. No. 62/613,202, filed on Jan. 3, 2018, the entire contents of which are hereby incorporated by reference in its entirety.

### FIELD OF THE INVENTION

The present invention relates to a pharmaceutical solid dosage forms and of solid emulsifying compositions of at least one cannabinoid, wherein the at least one cannabinoid is essentially solubilized in a mixture of at least one terpene the and at least two emulsifiers to form an oily phase which is adsorbed on a carrier. The composition self emulsifies upon contact with water and has sub-micron particle size and exhibits enhanced oral bioavailability and solubility and provides a convenient method of administration and lower dosages of the at least one active agent.

### BACKGROUND OF THE INVENTION

Cannabinoids have low oral absorption due to low solubility and high first pass effect. To overcome low oral bioavailability, various lipid-based drug delivery systems and self-emulsifying systems were developed. Lipid-based delivery systems and particularly self-emulsifying drug delivery systems (SEDDS) were demonstrated to increase the solubility, dissolution and bioavailability of many insoluble drugs. Presently, no solid self-emulsifying cannabinoid delivery system is US-marketed.

There is an unmet need for ready to use and patient-friendly oral cannabinoids dosage formsfororal, buccal, sublingual or intestinal absorption, whereas the dosage form will comprise the required cannabinoids in a patient friendly dosage form to maximize pharmacological efficacy and will present the insoluble cannabinoid in as small particle as possible and in a solubilized form that will improve the poor oral bioavailability the cannabinoids.

It has been unexpectedly discovered that cannabinoids, dissolved in terpenes or triglyceride oil or their mixture, and emulsifiers to form a “self emulsifying composition” which is absorbed onto an adsorbing powder, can form a solid dosage form that is self-emulsifying when in contact with body fluids and to produce a very fine sub-micron emulsion of plurality of particle with mean particle size of much below one micron and show significant improved oral bioavailability of at least 50%.

There remains an unmet need to provide cannabis oral medication with specific pharmacological effects, such as increase alertness, sedative to treat insomnia, antidepressant, anti-fatigue and pain relief in highly acceptable and convenient dosage form that will contribute to maximal patent compliance and enhanced oral absorption.

### SUMMARY OF THE INVENTION

The present invention relates to solid compositions, formulations and delivery systems, for the administration of combinations of at least one cannabinoid with at least one terpene or essential oil with at least two emulsifiers and optionally a triglyceride oil, adsorbed on solid sorbent to form a solid delivery system that self-smoltifies into nano-size plurality of particles upon contacts with mammals' body fluids and that provides improved oral absorption. More particularly, the present invention relates to compositions and dosage forms of cannabis or cannabinoids, terpenes, emulsifiers and adsorbing powder, which self emulsifies to have a plurality of particle in the sub-micron range, and improved oral bioavailability through mucus and the gastrointestine and has long shelf life stability.

The inventor has unexpectedly discovered that it is possible to have a solid dosage form that is emulsifiable cannabinoid composition, with desired cannabinoids, terpenes and emulsifiers and adsorbing powder, that has good shelf life stability and improved oral bioavailability. Such findings led to the discovery of the pharmaceutical compositions and formulations of the present invention, which are demonstrated herein to possess several therapeutically-beneficial properties. For example, the pharmaceutical, medicinal or veterinary compositions of the present invention comprise mixture of cannabinoids, terpenes, and optionally oils, emulsifiers and a sorbent, in a stable solid state, that emulsifies into very fine sub-micron and nano-size emulsion of oil-in-water type and may also comprise terpenes that are tailored and assembled for specific pharmacological need. Secondly, the compositions of the present invention are stable solids providing long shelf life stability at ambient room temperature of at least one year and for two years. More, the pharmaceutical compositions of the present invention are emulsified cannabinoid composition having very fine submicron droplets size, thus improving the oral bioavailability of the cannabinoids by at least 50% and more preferably by at least 100% or by at least 200%.

In certain embodiments, the composition comprises about 0.02% to about 10% by weight of a cannabinoid or a mixture of cannabinoids. In certain embodiments, the composition comprises about 0.05% to about 2% by weight of a cannabinoid or a mixture of cannabinoids. In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and mixtures of cannabinoids.

In certain embodiments, the composition comprises about 1% to about 25% by weight of a non-ionic emulsifier or a mixture of non-ionic emulsifiers with HLB>10 and HLB of about 10 to about 16. In certain embodiments, the composition comprises about 2% to about 10% by weight of an emulsifier or a mixture of emulsifiers. In certain embodiments, the emulsifier is selected from the group consisting of, sucrose stearate, sucrose distearate, sucrose laurate, sucrose palmitate, sucrose oleate, polysorbate, polysorbate 80, polyoxyl glycerides, polyoxyl 35 hydrogenated castor oil, tocopherol polyethylene glycol 1000 succinate, lauroyl polyoxyl-32 glycerides, polyglyceryl fatty acid esters, sorbitan fatty acid esters and mixtures of the emulsifiers.

In certain embodiments, the oily phase mixture of cannabinoids, terpenes, and emulsifiers and optionally oils, is adsorbed on a sorbent or adsorbing powder, and the obtained mixture is formed into granules or pellets of waxy solid nature.

In certain embodiments, the composition comprises from about 20% to about 90% of at least one sorbent, In certain embodiments, the composition comprises from about 30% to about 80% of at least one sorbent, In certain embodiments, the composition comprises from about 40% to about 70% of at least one sorbent, In certain embodiments, the composition comprises from about 40% to about 60% of at least one sorbent. In certain embodiments, the sorbent is selected from; silicon dioxide, colloidal silicon or fumed silica, inorganic sorbents such as synthetic Magnesium Aluminum, di- and tribasic calcium phosphates, calcium carbonate, calcium silicate, zeolites, talcite, kaolin, bentonite, microcrystalline cellulose (MCC), cross-linked polymers with high surface area, such as cross-linked povidone (PVO), and combinations or mixture thereof.

In certain embodiments, the pharmaceutical composition comprises about 1% to about 25% by weight of at least one triglyceride oil or a mixture of oils. In certain embodiments, the pharmaceutical composition comprises about 1% to about 10% by weight of an oil or a mixture of oils. In certain embodiments, the oil is selected from the group consisting of vegetable oils, such as; borage oil, coconut oil, cottonseed oil, soybean oil, safflower oil, sunflower oil, castor oil, hydrogenated castor oil, corn oil, olive oil, palm oil, peanut oil, peppermint oil, poppy seed oil, canola oil, soybean oil, hydrogenated soybean oil and sesame oil. In a certain embodiment a prefers triglyceride oils is capric/caprylic triglycerides which is a medium chain triglycerides (MCT). In a preferred embodiment a preferred oil is olive oil, fractionated palm oil, and sesame oil.

In certain embodiments, the composition of the emulsified cannabinoid has a plurality of particles having a mean particle size of 5 microns or less. In certain embodiments, the pharmaceutical composition of the emulsified cannabinoid has a plurality of particles having a mean particle size of 2 microns or less. In certain embodiments, the pharmaceutical composition of the emulsified cannabinoid has a plurality of particles having a mean particle size of 1 microns or less. In certain embodiments, the pharmaceutical composition of the emulsified cannabinoid has a plurality of particles having a mean particle size of 0.5 microns or less.

In certain embodiments, the dosage form is formulated as granules, pellets, micro-particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, toffee, chocolate or cookie. In certain embodiments, the tablet or pellets are an immediate release or slow or controlled release dosage dorms In certain embodiment the tablet is enteric coated or is a melt or dissolved in the mouth or is muco adhesive dosage form.

In certain embodiments, any one of the compositions described above, or any one of the dosage forms described above, is for use in a method of treating a cannabinoid-responsive symptom, disease or disorder.

In certain embodiments, the composition or dosage form comprises cannabidiol (CBD). In certain embodiments, the pharmaceutical composition or dosage form further comprises tetrahydrocannabinoid (THC). In certain embodiments, the CBD:THC weight ratio is about 20:1. In certain embodiments, the mixture comprises CBD. In certain embodiments, the mixture comprises THC. In certain embodiments, the mixture comprises CBD and THC. In certain embodiments, the mixture comprises CBD and THC in a weight ratio of about 1:1. In certain embodiments, the mixture comprises CBD and THC in a weight ratio of about 10:1 to about 1:10.

In certain embodiments, the pharmaceutical composition further comprises about 0% to about 30% by weight of fatty acid or fatty alcohol, glyceryl or propylene glycol mono or di stearate, fats, lipids, oils other than essential oils, co-solvents or mixtures thereof.

The method of production granules, or pellets, or tablets, compressed tablets, melt in mouth tablet, muco adhesive tablet. Enteric coating, immediate or slow release tablets and capsules and other dosage forms may be obtained from any standard reference work in this field, including, for example: Remington's Pharmaceutical Sciences, Mack Publishing Co, Easton, Pa., USA (1980).

### Definitions

The term “cannabinoid” as used herein generally refers to one of a class of diverse chemical compounds that act on a cannabinoid receptor in cells that repress neurotransmitter release in the brain. The term “cannabinoid” as used herein further refers a chemical compound that acts on cannabinoid receptors or has a structure similar the stature of a compound acting on cannabinoid receptor in cells. Ligands for these receptor proteins include the endocannabinoids (produced naturally in the body by humans and animals), the Phyto cannabinoids (found in cannabis and some other plants), and synthetic cannabinoids (manufactured artificially).

The term “cannabis extract” as used herein refers to one or more plant extracts from the cannabis plant. A cannabis extract contains, in addition to one or more cannabinoids, one or more non-cannabinoid components which are co-extracted with the cannabinoids from the plant material. Their respective ranges in weight will vary according to the starting plant material and the extraction methodology used. Cannabinoid-containing plant extracts may be obtained by various means of extraction of cannabis plant material. Such means include but are not limited to: supercritical or subcritical extraction with CO₂, extraction with hot or cold gas and extraction with solvents.

The term “essential oil” or “essential oils” as used herein relates to a concentrated hydrophobic liquid oil containing volatile aroma compounds, obtained from plants. Essential oils are also known as volatile oils, ethereal oils, due their distinct typical strong volatility at ambient temperature, or simply as the oil of the plant from which they were extracted, such as “oil of lavender”. An oil is “essential” in the sense that it contains the “essence of” the plant's fragrance—the characteristic fragrance of the plant from which it is derived.

Essential oils are generally extracted by distillation, often by using steam. Other processes include expression, solvent extraction, absolute oil extraction, resin tapping, and cold pressing. They are used in perfumes, cosmetics, soaps and other products, for flavoring food and drink, and for adding scents to incense and household cleaning products. The essential oils are comprised mainly of terpenes or terpenoids and the various properties of the different essential oils are derived from their terpenes content.

The term “terpene” as used herein also covers terpenoids. Terpenes are lipophilic compounds, volatile and liquid at room temperature and are used herein in this invention as part of the composition that contribute to increased bioavailability as well as cannabinoids solubilizers and as pharmacologically active agents that works in synergy or entourage with the cannabinoids. Terpenes are volatile organic compounds formed by the union of hydrocarbon of 5 carbon atoms, known as isoprene. The smallest and most volatile compounds are monoterpenes, which are biosynthesized by the union of two isoprene molecules. The biggest and least volatile are biosynthesized by the union of three or more isoprene molecules. The sesquiterpenes are next in the chain, which are formed by the union of three isoprene molecules. Terpenes are secondary metabolites, which provide the plant with its organoleptic characteristics (aroma and flavor) and that constitutes most of the essential oil produced by aromatic plants. Terpenes are major secondary metabolites of cannabis and are responsible for the odor and flavor of various cannabis strains. Cannabis strains and hemp strains produce many terpenes as secondary metabolites. Terpenes are synthetized from terpene unit into mono-terpenes, sesqui-terpenes, di-terpenes that are lipophilic, volatile and insoluble in water and are cyclic or bicyclic or not cyclic and may have alcohol, aldehyde or ketone chemical moiety. The term “terpene” further relates to essential oils. The term “terpene” does not include fats and/or lipids.

Generally, terpenes are derived biosynthetically from units of isoprene, which has the molecular formula C₅H₈. The basic molecular formula of terpenes are multiples of (C₅H₈)ₙ where n is the number of linked isoprene units. As chains of isoprene units are added, the resulting terpenes are classified sequentially by size as hemiterpenes, monoterpenes, sesquiterpenes, diterpenes, sesterterpenes, triterpenes, and tetraterpenes. Essentially, they are all synthesized by terpene synthase.

The term “about” as used herein refers to any value which lies within a range of ±5% of original value. For example, “about 100” refers to “95 to 105”.

The term “emulsifier” as used herein are amphiphilic molecules that are surface active, or surfactants, and that stabilize emulsions by reducing the interfacial tension.

The term “triglyceride oil” is used here is an oil made of three fatty acids conjugated by an esters bond to glycerin or glycerol, whereas the fatty acids are having from C8 to C22 carbons. The fatty acids may be unsaturated or saturated and hydrogenated or not. The triglyceride oil may also comprise diglycerides and free fatty acids in small amount. The triglyceride can be liquid or solid at room temperature. A preferred type of triglyceride is capric/caprylic triglyceride that is less prone to oxidation.

The term “solid self-emulsifying” or “solid self-emulsifying composition” or “self-emulsified cannabinoids compositions” means any cannabinoid that is fully dissolved in the mixture of at least one triglyceride oil and at least one emulsifier and absorbed onto adsorbing powder to form a solid composition, and whereas the “solid self-emulsifying composition” emulsifies into a sub-micron emulsion of oil-in-water type. The cannabinoid may be an oily extract of cannabis type plant, or pure cannabinoid or synthetic or cannabinoid derivative, dissolved in a vegetable oil, or pure cannabinoid dissolved in a mixture of triglyceride oils and at least one emulsifier, thus enabling high cannabinoid load above the maximal concentration of a cannabinoid in a specific triglyceride oil. The maximal concentration of a specific cannabinoid in any triglyceride oil, and same for maximal solubility of a specific cannabinoid in a solution of a triglyceride oil and emulsifier, is higher than in the vegetable oil alone.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The term “excipient” as used herein has its conventional meaning and refers to a pharmaceutically acceptable ingredient, which is commonly used in the pharmaceutical technology for preparing a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes. The term “veterinary composition” encompasses the full range of compositions for internal administration and feeds and drinks which can be consumed by animals.

As used herein, the term “particles” as used herein relates to droplets. The term “particle size” of an emulsion is to be understood also as the “droplet size” of that emulsion. The term “mean particle size” is also to be understood as the term “mean droplet size”.

As used herein, the term “mean particle size” refers to a value which is obtained by measuring the diameters in a specific direction of particles and dividing the sum of respective diameters of particles by the number of measured particles.

As used herein, the term “essentially solubilized” means that at least 80%, or 90% or 95% or 98% or 99% of the cannabinoids is molecularly dissolved in the composition or in a specific part or excipients of the composition.

The methods, uses, materials, and examples that will now be described are illustrative only and are not intended to be limiting; materials, uses and methods similar or equivalent to those described herein can be used in practice or testing of the invention. Other features and advantages of the invention will be apparent from the following figures, detailed description, and from the claims.

### BRIEF DESCRIPTION OF THE DRAWINGS

FIG. 1. Light microscope magnification ×400 of formulation 1A mixed gently with water ˜1:10 to produce a dispersion of a) the sorbent (S) and b) a fine oil-in-water sub micron emulsion.

FIG. 2. Picture of a solid pellete and pellete disperseed in simulated intestinal fluids (SIF) to produce a dispersion of a) the sorbent and b) a fine oil-in-water sub micron emulsion.

### DETAILED DESCRIPTION OF THE INVENTION

Provided by the present invention are compositions of solid emulsified cannabinoids of at least one cannabinoid and at least one terpene or essential oil and at least two emulsifiers and at least one adsorbing powder, that are producing a sub-micron emulsion and nano-size emulsion upon contact with mammals body fluids, and having increased oral bioavailability in comparison to the same cannabinoid dissolved in organic solvent or mixture of organic solvents, and over one-year stability at ambient room temperature.

Without being limited to any theory or mechanism, the present invention is based on the surprising finding that a composition of a: at least one cannabinoid and b: at least one terpene or essential oil, c: at least two emulsifiers, d: at least one adsorbing powder, wherein the solid is self emulsifies in aqueous medium, and are exceptionally proficient, in producing stable solid cannabis product and dosage forms that are highly convenient to consume and increase the patient compliance and thus the pharmacological effect, and achieving high oral bioavailability which improve the efficacy or enable use of a lower dose.

It has been unexpectedly discovered that a composition of at least one cannabinoid dissolved in a mixture of at least one triglyceride oil or at least one terpene, at least one essential oil or combinations thereof, and at least two non-ionic emulsifiers, all this ingredients or components together are termed the “oily phase” or “self emulsifying composition”, that is adsorbed onto an at least one adsorbing powder, the “sorbent”, can produce a solid composition, that can be granulated and/or compressed into a tablet, and thus produce a plurality of particles of below one micron, upon contact with water or with mammal's body fluids and increase the at least one cannabinoid oral bioavailability by at least 50% or by at least 100% or by at least 200% in comparison to the same cannabinoid dissolved in a solvent or solvents mixture, such as propylene glycol and ethanol.

It has been unexpectedly discovered that when the solid self emulsifying composition disintegrates and dissolves in gastro-intestinal fluids, it produces a very fine nano-emulsions or sub-micron emulsions of the oil-in-water type comprising cannabinoids, that are produced in a specific set of conditions and compositions, whereas the at least two emulsifiers are selected, having both an HLB of 10 to 16 or at least one of them have an HLB of 10 to 16 and preferably one of the emulsifier is selected form sucrose fatty acid esters; such as sucrose stearate, tocopheryl polyethylene glycol 1000 succinate, polysorbate, polyglyceryl fatty acid esters, polyoxyl hydrogenated castor oil and the second emulsifier is selected from HLB of non-ionic emulsifiers of HLB 4 to 15 selected from sucrose fatty acid esters; such as sucrose stearate, tocopheryl polyethylene glycol 1000 succinate, sorbitan fatty acid esters, polysorbate, polyglyceryl fatty acid ester, polyglyceryl-3 dioleate and polyoxyl hydrogenated castor oil.

The present invention provides, in one aspect, a solid emulsified composition, comprising: 1) about 0.02% to about 10% by weight of a cannabinoid or a mixture of cannabinoids, and 2) about (a) 1% to about 20% of a triglyceride oil, or (b) from about 0.5% to about 5.0% of at least one terpen or essential oil, and 3) about 1% to about 10% by weight of an emulsifier or a mixture of emulsifiers, and 4) about 20% to about 90% by weight of an adsorbing powder or a mixture of adsorbing powders, wherein upon contact and mixing with mammals body fluids, the solid composition is transformed into a dispersion comprising of: a) the sorbent that is water insoluble and b) an oil-in-water emulsion type, that has a plurality of oily particles having a mean particle size of about 10 nm to about 2,000 nm and more preferably from about 100 nm to about 1000 nm or from 100 nm or from 800 nm to about 100 nm and from 600 nm to about 50 nm and from 400 nm or from about 10 nm to about 200 nm.

The present invention provides, in one aspect, is a solid emulsified composition, comprising: about 0.02% to about 10% by weight of a cannabinoid or a mixture of cannabinoids, and about 1% to about 20% of an oil, and about 1% to about 10% by weight of an emulsifier or a mixture of emulsifiers, about 20% to about 90% by weight of an adsorbing powder or a mixture of adsorbing powders, wherein the ratio of the at least one oil to at least one emulsifier is about 1:10 to about 5:1 and more preferably from about 1:5 to about 2:1 and more preferably from about 1:2 to 3:2, and wherein the composition has a plurality of particles having a mean particle size of about 10 nm to about 2,000 nm and more preferably from 10 nm to 1,000 nm and more preferable from about 10 nm to 800 nm or from 100 nm to 600 nm.

The solid composition self-emulsifies instantaneously into an oil-in-water emulsion having very fine opaque to translucent emulsion. The sub-micron or nano-size range of the oil internal phase droplets is from about 10 nm to about 1,000 nm and from about 10 nm to about 800 nm and more particularly from about 20 nm to about 600 nm or from about 30 nm to about 400 nm or from about 40 nm to about 300 nm or from about 50 nm to about 200 nm.

In certain embodiments “solid emulsified composition” may be produced by heating the cannabinoid, with the terpenes, essential oils, oils and the emulsifier to about 80° C., and mix until homogeneous and mixing with the adsorbing powder and with the other ingredients, optionally under high speed planetary mixer or granulator or a roller miller and compactor machines, as known in the pharmaceutical art.

In certain embodiments, the at least one “absorbing powder” or “sorbent” is an organic or non-organic powder with large porosity and surface area which is used in the art to absorb liquids. In certain embodiments, the absorbing powder is selected from, silicon dioxide, colloidal silicon (dried silicagel, Syloid™ 244, GRACE; Sipernats™, DEGUSSA) or fumed silica (prepared by hydrolysis of silicone alides -Cab-O-Sil™ M5, CABOT, or Aerosil™ 200/300, DEGUSSA), inorganic sorbents such as synthetic Magnesium Aluminum Silicate (Neusilin™, FUJI), di- and tribasic calcium phosphates, calcium carbonate, calcium silicate, zeolites, talcite, kaolin, bentonite, microcrystalline cellulose (Avicel™ 611), cross-linked polymers with high surface area, such as cross-linked povidone (Povidone™ XL, BASF) as an examples.

The sorbent function is to hold the lipid phase during the granulation process to provide free flowing granulation, and prevent the lipid phase from leaking during the tableting process. The sorbent should be physiologically inert, safe and suitable for granulation and tableting processes. The sorbent should possess high surface area/porosity, high mechanical strength.

In certain embodiments, the pharmaceutical composition comprises about 0.01% to about 20% by weight of a cannabinoid or a mixture of cannabinoids. In certain embodiments, the pharmaceutical composition comprises about 0.02% to about 10% by weight of a cannabinoid or a mixture of cannabinoids. In certain embodiments, the pharmaceutical composition comprises about 0.05% to about 5% by weight of a cannabinoid or a mixture of cannabinoids. In certain embodiments, the pharmaceutical composition comprises about 0.1% to about 2% by weight of a cannabinoid or a mixture of cannabinoids.

In certain embodiments, the cannabinoid is a natural cannabinoid. In certain embodiments, the cannabinoid is a natural cannabinoid found in a Cannabis plant. In certain embodiments, the cannabinoid is a synthetic cannabinoid. In certain embodiments, the cannabinoid is a mixture of natural cannabinoids. In certain embodiments, the cannabinoid is a mixture of synthetic cannabinoids. In certain embodiments, the cannabinoid is a mixture of natural and synthetic cannabinoids.

The term “natural cannabinoid” as used herein generally refers to a cannabinoid which can be found in, isolated from and/or extracted from a natural resource, such as plants. “Synthetic cannabinoids” are a class of chemicals that are different from the cannabinoids found e.g. in cannabis but which also bind to cannabinoid receptors.

In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

The terms “cannabidiol” and “CBD” are interchangeably used herein and refer to a non-psychotropic cannabinoid having structure as described in Formula I below, salt or derivatives thereof, such as Δ⁴-cannabidiol, Δ⁵-cannabidiol, Δ⁶-cannabidiol, Δ^{1,7}-cannabidiol, Δ¹-cannabidiol, Δ²-cannabidiol, Δ³-cannabidiol.

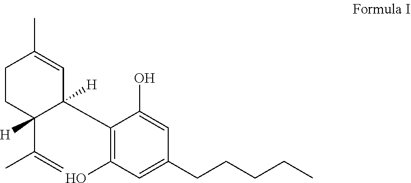 [SMILES: C=C(C)[C@@H]1CCC(C)=C[C@H]1c1c(O)cc(CCCCC)cc1O]

In another embodiment, the pharmacologically active cannabinoid may be selected from the group consisting of tetrahydrocannabinol, A9-tetrahydrocannabinol (THC), A8-tetrahydrocannabinol, standardized marijuana extracts, A8-tetrahydrocannabinol-DMH, A9-tetrahydrocannabinol propyl analogue (THCV), 11-hydroxy-tetrahydrocannabinol, 11-nor-9-carboxy-tetrahydrocannabinol, 5′-azido-.Δ8-tetrahydrocannabinol, AMG-1 (CAS Number 205746-46-9), AMG-3 (CAS Number 205746-46-9), AM-411 (CAS Number 212835-02-4), (−)-11-hydroxy-7′-isothiocyanato-A8-THC (AM-708), (−)-11-hydroxy-7′-azido-A8-THC (AM-836), AM-855 (CAS Number 249888-50-4), AM-919 (CAS Number 164228-46-0), AM926, AM-938 (CAS Number 303113-08-8), cannabidiol (CBD), cannabidiol propyl analogue (CBDV), cannabinol (CBN), cannabichromene, cannabichromene propyl analogue, cannabigerol, CP 47,497 (CAS Number (1S,3R): 114753-51-4), CP 55,940 (CAS Number 83002-04-4), CP 55,244 (CAS Number 79678-32-3), CT-3 (ajulemic acid), dimethylheptyl HHC, HU-210 (1,1-Dimethylheptyl-11-hydroxy-tetrahydrocannabinol), HU-211 (CAS Number 112924-45-5), HU-308 (CAS Number 1220887-84-2), WIN 55212-2 (CAS Number 131543-22-1), desacetyl-L-nantradol, dexanabinol, JWH-051 (Formula C₂₅H₃₈O₂), levonantradol, L-759633 (Formula C₂₆H₄₀O₂), nabilone, O-1184, and mixtures thereof.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; and enol forms.

Extraction of cannabis plant of various plant parts, may be done by CO₂ extraction or by solvent extraction or solvent-less compression to obtain oily viscous material or waxy material or solid material, depends on plant material, plant parts and extraction methods as skilled in the art. Extraction and processing may result in broad spectrum of cannabis molecules, cannabinoids, terpenes and other families of natural cannabis molecules or in a pure extract of cannabinoids or concentrated cannabinoid terpenes extract. Cannabis or marijuana extract may be further decarboxylated, winterized and/or purified, for example by distillation, as known in the art.

In certain embodiments, the pharmaceutical composition comprises about 0.01% to about 5% by weight of a terpene or a mixture thereof. In certain embodiments, the pharmaceutical composition comprises about 0.05% to about 4% by weight of a terpene or a mixture thereof. In certain embodiments, the pharmaceutical composition comprises about 0.05% to about 1% by weight of a terpene or a mixture thereof. In certain embodiments, the pharmaceutical composition comprises about 0.5% to about 4% by weight of a terpene or a mixture thereof. In certain embodiments, the pharmaceutical composition comprises about 0.5% to about 3% by weight of a terpene or a mixture thereof. In certain embodiments, the pharmaceutical composition comprises about 1% to about 2% by weight of a terpene or a mixture thereof.

In certain embodiments, the pharmaceutical composition comprises about 0.01% to about 20% by weight of a mixture of a cannabinoid and a terpene. In certain embodiments, the pharmaceutical composition comprises about 0.02% to about 10% by weight of a mixture of a cannabinoid and a terpene. In certain embodiments, the pharmaceutical composition comprises about 0.05% to about 5% by weight of a mixture of a cannabinoid and a terpene. In certain embodiments, the pharmaceutical composition comprises about 0.5% to about 2% by weight of a mixture of a cannabinoid and a terpene. In certain embodiments, the pharmaceutical composition comprises about 0.001% to about 1% by weight of a mixture of a cannabinoid and a terpene. In certain embodiments, the pharmaceutical composition comprises about 0.05% to about 0.5% by weight of a mixture of a cannabinoid and a terpene.

In certain embodiments of the pharmaceutical composition, the weight ratio between the cannabinoid and the terpene is about 50:1 to about 1:1. In certain embodiments of the pharmaceutical composition, the weight ratio between the cannabinoid and the terpene is about 20:1 to about 1:1. In certain embodiments of the pharmaceutical composition, the weight ratio between the cannabinoid and the terpene is about 15:1 to about 1:1. In certain embodiments of the pharmaceutical composition, the weight ratio between the cannabinoid and the terpene is about 10:1 to about 1:1. In certain embodiments of the pharmaceutical composition, the weight ratio between the cannabinoid and the terpene is about 5:1 to about 1:1.

In certain embodiments, the composition comprises an essential oil or a terpenes or combinations thereof. In certain embodiments, the terpene is a natural terpene found in a Cannabis plant. In certain embodiments, the terpene is a synthetic terpene. In certain embodiments, the terpene is a mixture of natural terpenes. In certain embodiments, the terpene is a mixture of synthetic terpenes. In certain embodiments, the terpene is a mixture of natural and synthetic terpenes.

In certain embodiments, the terpene is selected from the group consisting of bisabolol, borneol, caryophyllene, carene, camphene, cineol, citronella, eucalyptol, geraniol, guaiol, humulene, isopropyltoluene, isopulegol, linalool, limonene, methyl salicylate, menthol, myrcene, nerolidol, ocimene, pinene, phytol, pulegone, terpinene, terpinolene, thymol, salts thereof, derivatives thereof and mixtures thereof. Each possibility represents a separate embodiment of the invention.

In certain embodiment the terpene is a cannabis plant terpene, or a terpene derived from a non-cannabis plant material or a synthetic terpene. In certain embodiment the terpene is a taste modifier or smell modifier agent, a food grade or pharmaceutical grade, a solubilizer or solvent and an excipient in the formulation.

120 distinct terpenes are produced by the genus Cannabis, with the relative concentrations of the individual terpenes varying greatly among the 700 distinct strains currently in cultivation. Aside from taste and smell differences between varieties, this helps contribute to the broad diversity of potential medical applications of Cannabis.

In certain embodiments of the pharmaceutical composition, the natural cannabinoid is derived or isolated from an extract of a Cannabis plant. In certain embodiments of the composition, the natural terpene is derived or isolated from an extract of a Cannabis plant.

In certain embodiments, a terpene or the mixture of terpenes solubilized with a cannabinoid or a mixture of cannabinoid. Each possibility represents a separate embodiment of the invention.

In certain embodiments of the pharmaceutical composition, the weight ratio between the cannabinoid and the emulsifier is about 1:10 to about 2:1. In certain embodiments of the pharmaceutical composition, the weight ratio between the cannabinoid and the emulsifier is about 1:8 to about 1:1. In certain embodiments of the pharmaceutical composition, the weight ratio between the cannabinoid and the emulsifier is about 1:5 to about 1:1. In certain embodiments of the pharmaceutical composition, the weight ratio between the cannabinoid and the emulsifier is about 1:2 to about 1:1.

The emulsifier ingredients of the composition of this invention improve the cannabinoid solubilization and the emulsifying properties of the formulation. The emulsifiers, also termed surfactants, are selected from the group consisting of polyglycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glycerol esters of saturated C8-C18 fatty acids (Gelucire® 33/01), glyceryl esters of saturated C12-C18 fatty acids (Gelucire® 39/01 and 43/01), glyceryl laurate/PEG-32 laurate (Gelucire® 44/14), glyceryl laurate glyceryl/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), lecithin, phospholipids and mixtures thereof. Polyglycolized derivatives and polyoxyethylene esters or ethers derivatives of medium to long chain fatty acids, commercially named Brij and Myrj variety surfactants, and propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, glyceryl caprylate/caprate, medium chain (C8/C10) mono- and diglycerides (Capmul® MCM, Capmul® MCM (L)), mono- and diacetylated monoglycerides, polyglyceryl oleate, polyglyceryl-2 dioleate, polyglyceryl-10 trioleate, polyglyceryl-10 laurate, polyglyceryl-10 oleate, and polyglyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, and mixtures thereof. Sucrose esters surfactants such as sucrose stearate, sucrose distearate, sucrose palmitate, sucrose oleate and polyethylene glycol sorbitan fatty acid esters, which can be used, include PEG-20 sorbitan monolaurate, PEG-20 sorbitan monopalmitate, PEG-20 sorbitan monostearate, and PEG-20 sorbitan monooleate, and TPGS (d-.alpha.-tocopheryl polyethylene glycol 1000 succinate), polysorbate 20 (Tween® 20), polysorbate 80 (Tween® 80), polyethyleneglycol 660 12-hydroxystearate (Solutol® HS-15 or Kolliphor® HS15), polyglyceryl-3 dioleate (Plurol® Oleique CC 497, Gattfosse), Acrylates/C10-30 Alkyl Acrylate Cross-Polymer (Pemulene™ TR2, Lubrizol). sodium lauryl sulfate and combinations thereof.

Polyoxyethylene-polyoxypropylene block copolymers, which can be used as emulsifiers in the compositions of this invention include poloxamers (108, 124, 182, 183, 188, 212, 217, 238, 288, 331, 338, 335, and 407), and mixtures thereof. Sorbitan fatty acid esters, which can be used, include sorbitan monolaurate (Span® 80), sorbitan monopalmitate, sorbitan monooleate (Span® 20), sorbitan monostearate, sorbitan tristearate and combinations thereof.

A preferred type of emulsifiers are polymeric surfactant, such as Acrylates/C10-30 Alkyl Acrylate Cross-Polymer. Or alkyl acrylate cross polymer, Pemulen™ TR1 or TR2, from Lubrizol.

In certain embodiments, the composition comprises from about 0.5% w/w to about 25% w/w of at least two emulsifiers. In certain embodiments, the pharmaceutical composition comprises from about 1% w/w to about 15% w/w of at least two emulsifiers. In certain embodiments, the two emulsifiers are selected from the group consisting of polysorbate, polysorbate 80, polyoxyl 35 hydrogenated castor oil, sucrose fatty acids esters such as, sucrose stearate and sucrose distearate, tocopherol polyethylene glycol 1000 succinate, polyglyceryl-3 dioleate, sorbitan monooleate, sorbitan monoleate, sucrose tatty acid ester, polyglyceryl fatty acid ester and alkyl acrylate cross polymer and salts thereof, derivatives thereof and combinations thereof.

In certain embodiments, a mixture of at least two emulsifiers is more effective in providing stable and low particle size of emulsified and solid cannabinoid composition. In a preferred embodiment, at least one of the emulsifiers is a sucrose fatty acid ester selected from sucrose stearate, sucrose distearate, sucrose oleate, sucrose palmitate, sucrose laurate, sucrose tetrastearate and sucrose polystearate. Preferred sucrose esters are sucrose stearates for example Surfhope™ D-1811F, Surfhope™ D-1815, Surfhope™ D-1615 all from Mitsubishi chemicals, Crodesta™ F110 which is a mixture of mono and diesters with a HLB value of 12, or Crodesta™ F160 which is a monoester with a HLB value of 14.5.

In certain embodiments the at least two surfactants or emulsifiers are selected from; sucrose ester, PEG-Castor oil, TPGS, Polysorbate, polyglyceryl fatty acid esters, such as polyglyceryl-3 dioleate, and sorbitan fatty acid esters such as sorbitan laurate.

In certain embodiments, the at least one surfactant having HLB of about 2 to about 12, is selected from and selected from polyglyceryl-3 dioleate HLB=3, span 80 HLB=4.3, span 85 HLB=1.8, span 60 HLB=4.7, span 40 HLB=6.7, span 83 HLB=3.7, span20=8.6, Sorbitan Oleate HLB=4.3, Sorbitan Monostearate NF HLB=4.7, Sorbitan Stearate HLB=4.7, Sorbitan Isostearate HLB=4.7, Steareth-2 HLB=4.9, Oleth-2 HLB=4.9, Glyceryl Laurate HLB=5.2, Ceteth-2 HLB=5.3, sucrose tristearate HLB=3, sucrose distearate HLB=6, sucrose sterate (50% mono stearate) HLB=11, sucrose tribehenate HLB=3.

In certain embodiments of this invention, the two emulsifiers are selected from, polysiorbates, tocopheryl PEG succinate, polyoxyl castor oil, sucrose esters and sorbitan esters, polyglyceryl fatty acid esters and combinations thereof and a third emulsifier is selected from Acrylates/C10-30 Alkyl Acrylate Cross-Polymer.

In certain embodiments the HLB of the at least one emulsifier is about HLB 8 to about HLB 18, In certain embodiments the HLB of the at least one emulsifier is about HLB 8 to about HLB 16, In certain embodiments the HLB of the at least one emulsifier is about HLB 10 to about HLB 16, In certain embodiments the HLB of the at least one emulsifier is about HLB 8 to about HLB 15, In certain embodiments the HLB of the at least one emulsifier is about HLB 11 to about HLB 16, In certain embodiments the HLB of the at least one emulsifier is about HLB 11 to about HLB 15, In certain embodiments the HLB of the at least one emulsifier is about HLB 11 to about HLB 14. Each possibility represents a separate embodiment of the invention.

In some preferred embodiments the average HLB calculated on the molar basis HLB of the mixture of the emulsifiers is from about HLB 6 to about HLB 16. In certain embodiments the combined HLB calculated on the molar basis HLB of the mixture of the emulsifiers is from about HLB 8 to about HLB 16. In certain embodiments the combined calculated on the molar basis HLB of the emulsifier mixture is from about HLB 8 to about HLB 15. In certain embodiments the combined HLB calculated on the molar basis HLB of the mixture of the emulsifiers is from about HLB 10 to about HLB16. In certain embodiments the combined calculated on the molar basis HLB of the mixture of the emulsifiers is from about HLB 10 to about HLB 15. In certain embodiments the combined HLB calculated on the molar basis HLB of the mixture of the emulsifiers is from about HLB 10 to about HLB 14. In certain embodiments the combined HLB calculated on the molar basis HLB of the mixture of the emulsifiers is from about HLB 8 to about HLB 13. In certain embodiments the combined HLB calculated on the molar basis HLB of the mixture of the emulsifiers is from about HLB 10 to about HLB 12.

In certain embodiments, the pharmaceutical composition comprises (i) about 1% to about 20%, preferably about 2% to about 15%, more preferably about 2% to about 10% by weight of a cannabinoid or a mixture of cannabinoids; (ii) about 0.1% to about 20%, preferably about 1% to about 15%, and preferably 2% to 10%, of a solvent to the cannabinoid, selected from terpenes, essential oils and triglycerides: and (iii) about 1% to about 20% or about 2% to about 15% by weight of an emulsifier or a mixture of emulsifiers; and (iv) about 20% to about 90% by weight of a sorbent, and more preferably about 30% to about 80% by weight of a sorbent, and more preferably from about 40% to about 70% of a sorbent or a mixture of sorbents. Each possibility represents a separate embodiment of the invention.

In certain embodiments, the composition is formed into granules or pellets or microparticles and in certain embodiment the granules or pellets or micro particles are coated for taste masking or for improving chemical stability or enteric-coated.

In certain embodiment the composition is formed into a tablet, mixing with tablet forming materials, such as, glidants, lubricants, hydration regulators can be selected according to desired tablet properties and loading level. The tablet possesses desired physical characteristics such as hardness, friability, dissolution behavior and can be manufactured using standard equipment such as granulators, ovens, dryers, mixers, tablet press, drum coater, and the like as known in the art of pharmaceutical sciences. Upon contact with water or mammals body fluid the tablet releases the “oily phase”, forming an oil-in-water emulsion comprising an active cannabinoid dissolved in an oil phase and having plurality of particles of below one micron.

In certain embodiments the composition comprises a release controlling agent to produce a slow or prolonged or control the release of the at least one cannabinoid. A release controlling agent is selected from a polymer that control the dissolution rate of the solid dosage form, tablet or granule or pellet. Preferred polymers are water swellable or water soluble cellulose derivatives, for example, Hydroxypropyl methylcellulose (Methocel™, types A, E, K, F, Dow Chemical), Hydroxyethyl cellulose (Natrosol™, Hercules), Hydroxypropyl cellulose (Klucel™, Aqualon), Carboxymethylcellulose (cellulose gum). Another types of synthetic polymers include polyacrylic acid (Carbopol™, BFGoodrich), Polyethylene oxide (Polyox™, Union Carbide), Polyvinyl pyrrolidone (Kollidon™, PVP and PVP-VA, BASF), natural gums and polysaccharides-Xanthan gum (Xantural™, Kelco), carrageenan, locust bean gum, acacia gum, chitosan, alginic acid, hyaluronic acid, pectin, etc.

Without wishing to be bound to theory, a possible mechanism of slow release is the hydration and gelling of the polymers, in parallel with emulsification process. Release of the formed emulsion from the gel is by dissolution and partial diffusion of the sub-micron lipid droplets from gelled matrix to surrounded media by mechanism of solid dosage form erosion, disintegration or both.

In certain embodiments, the solid pharmaceutical composition of the cannabinoid self emulsifies into an oil-in-water very fine sub-micron emulsion, at body temperature and body fluids, having a plurality of particles having a mean particle size in the range of about 10 nm to about 2,000 nm. In certain embodiments, the pharmaceutical emulsified cannabinoid composition having of particles having a mean particle size by number in the range of about 10 nm to about 1,000 nm. In certain embodiments, the pharmaceutical emulsified cannabinoid composition having produce a plurality of particles having a mean particle size in the range of about 50 nm to about 800 nm. In certain embodiments, the pharmaceutical emulsified cannabinoid composition having produce a plurality of particles having a mean particle size in the range of 50 nm to about 600 nm or about 50 nm or about 400 nm or from about 50 nm to about 200 nm, of have plurality of particle populations.

In certain embodiments, the emulsified cannabinoid composition has a plurality of particles, wherein at least 90% of the particles have a size of 2 microns or less. In certain embodiments, the emulsified cannabinoid composition has a plurality of particles, wherein at least 90% of the particles have a size of 1 microns or less. In certain embodiments, the emulsified cannabinoid composition has a plurality of particles, wherein at least 95% of the particles have a size of 1 microns or less. In certain embodiments, the emulsified cannabinoid composition has a plurality of particles, wherein at least 98% of the particles have a size of 1 microns or less. In certain embodiments, the emulsified cannabinoid composition has a plurality of particles, wherein at least 99% of the particles have a size of 1 microns or less. In certain embodiments, the emulsified cannabinoid composition has a plurality of particles, wherein at least 99% of the particles have a size of 0.8 microns or less or 0.6 microns or less, or 0.4 microns or less.

Cytochrome P450/P-gp (PGP) inhibitors include any agent incorporated into the formulation matrix that inhibits pre-systemic hepatic first pass metabolism (i.e. first pass metabolism), such as d-.alpha.-tocopheryl polyethylene glycol 1000 succinate, anise oil, cinnamon oil, coriander oil, grapefruit oil, lemon oil, orange oil, peppermint oil, ascorbyl palmitate, propyl gallate, piperin, curcumin, resveratrol, terpenes (essential oils) and various combinations thereof.

Intestinal PGP efflux inhibitors include any agent incorporated into the formulation matrix that inhibits PGP induced cellular efflux mechanisms (i.e. MDR), such as polyethoxylated castor oil derivatives, polyoxyethylene sorbitan monooleate, polyoxyethylene glycerides, herbal extracts such as, for example; piperin, ginger licorice, berberin, terpenes (essential oils) and various combinations thereof.

Absorption enhancers are selected from herbal extracts such as piperin, ginger extract, berberin, liquorish, quercetin, resveratrol, terpenes and vitamin E PEG 1000 succinate (d-.alpha.-tocopheryl polyethylene glycol 1000 succinate or TPGS) and mixtures thereof.

These optional components can be used either alone or in combination with other ingredients to improve the chemical and physical properties of the self-emulsifying drug delivery systems and the cannabinoids or the terpenes chemical stability and shelf life.

Furthermore, the dosage form may include viscosity modifying agents, stabilizing agents, fillers, glidants disintegration agent, coating and enteric-coating, microbial preserving agents, buffers, taste masking agents, as skilled in the art to produce desired dosage form and manufacturability.

Antioxidants include ascorbyl palmitate, butylated hydroxy anisole, butylated hydroxy toluene, propyl gallate, α-tocopherol, and γ-tocopherol, etc. The antioxidants that can be chosen include combinations of two or more agents described above, whereby ascorbyl palmitate and tocopherol provide optimal synergistic effects.

The present invention further provides, in an aspect, a dosage form, comprising or consisting of any one of the compositions described above.

The term “dosage form” denotes any form of the solid formulation that contains an amount of a cannabinoid or of a mixture of cannabinoids sufficient to achieve at least a partial therapeutic effect with a single administration.

In certain embodiments, the dosage form is an oral dosage form. In certain embodiments, the dosage form is a rectal dosage form. In certain embodiments, the dosage form is a nasal dosage form. In certain embodiment, the dosage form is mucosal dosage form. In certain embodiments, the dosage form is a rectal or vaginal dosage form. In certain embodiments, the dosage form is a topical dosage form.

In certain embodiments, the dosage form is formulated as a tablet, enteric coated tablet, enteric coated capsule, dissolve in mouth tablet, dissolve in mouth strip, or capsule, enteric coated granules, granules or pellets. Each possibility represents a separate embodiment of the invention. In certain embodiments, the dosage form is formulated for mucosal delivery. The term “mucosal delivery” refers to the delivery to a mucosal surface, including nasal, vaginal, rectal, urethral, sublingual and buccal delivery. In certain embodiments, the dosage form is formulated in a candy, toffee, dragee, chocolate, cookie or lozenge.

According to the principles of the present invention, the emulsified cannabinoid compositions dosage form comprises a “cannabis active ingredient” (i.e. Cannabis-extracted and purified cannabinoid or synthetic cannabinoid or cannabis extract), a terpene or an oil, and an emulsifier or emulsifiers, adsorbing powder or adsorbing powders and optionally inactive ingredients. The advantages of the pharmaceutical composition over known cannabis compositions are manifold and include: (a) high oral bioavailability (b) stable solid self-emulsifying product enabling stable formulations and mixtures of desired cannabinoids and terpenes, in simple and common production methods and machinery (c) high dose of cannabinoid in a concise size dosage form and (d) a ready to use and easy to swallow and administer to all segments of population including infants, toddlers and elderly, for successful patient compliance and effective medication.

In some embodiments, a functional inactive ingredient maybe optionally added, such as colorant or antioxidants or chelating agent or microbial preservative or viscosity modifier, pH modifying agents, buffer, or melting point modifier or anti-microbial agent or suspending agent.

The present invention further provides, in another aspect, a composition as described above, or a dosage form as described above, for use in a method of treating a cannabinoid-responsive symptom, disease or disorder.

The phrase “cannabinoid-responsive symptom, disease or disorder” as used herein refers to any symptom, disease or disorder which is associated with therapeutic benefit by a cannabinoid, by a mixture of cannabinoids, or by extracts of Cannabis.

In certain embodiments, the cannabinoid-responsive symptom, disease or disorder is selected from the group consisting of: pain associated with cancer, neuropathic pain and HIV-associated sensory neuropathy; side effects of chemotherapy including nausea; symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anxiety, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, insomnia, fatigue, sleep apnea, digestive diseases, inflammatory bowel diseases, collagen-induced arthritis, atherosclerosis, auto immune diseases and dystonia and geriatric syndromes. Each possibility represents a separate embodiment of the invention.

People with PTSD may also benefit from marijuana use when it comes to sleep. When a person has experienced a traumatic event, they may suffer from nightmares that interfere with their sleep. Marijuana use is common among those with PTSD, and patients report using it to help them sleep. Using a synthetic THC as a treatment for PTSD-related nightmares, found that patients slept longer, had higher sleep quality, and experienced fewer daytime flashbacks when using the drug.

Another common reason people seek relief with cannabis is to reduce pain at night and improve sleep. The combination of pain and poor sleep can have serious effects on a person's wellbeing. Marijuana is known for its ability to relieve pain and to help with insomnia. Likewise, research shows that many people who use marijuana for chronic pain find that it also improves their quality of sleep.

Parkinson's disease is associated with a very unusual form of sleep disturbance. The disturbances are collectively referred to as REM sleep behavior disorder. During REM sleep, our bodies lose muscle tone which prevents us from physically acting out our dreams in bed. However, in REM sleep behavior disorder, the body no longer loses muscle tone, causing patients to act out their dreams. This may involve punching, pushing, yelling and swearing while sleeping. In a series of case studies, doctors observed improvements in patients who used CBD. They found that doses of 75-300 mg of CBD taken daily could reduce or eliminate entirely any symptoms of REM sleep behavior disorder.

Cannabis strains high in Myrcene and Linalool, usually of Indica type, are associated with sedation and better sleep.

Chronic pain is one of the most common conditions treated with medical marijuana. Similar to over-the-counter painkillers such as aspirin and ibuprofen, marijuana can reduce inflammation and pain associated with inflammation.

THC is the compound believed to reduce pain. It has been found to be effective in a variety of conditions that cause pain, including arthritis, migraine, multiple sclerosis and cancer. A 2015 clinical review examined 6 different trials with a total of 325 patients, and concluded that marijuana can be an effective treatment for patients with chronic pain. Hill at al, 2015, JAMA. 2015; 313(24):2474-2483. Medical Marijuana for Treatment of Chronic Pain and Other Medical and Psychiatric Problems. Hill at al, Cannabis and Pain: A Clinical Review, Cannabis and Cannabinoid Research Volume 2.1, 2017 Marijuana may be beneficial to those with certain liver disorders. In the case of liver fibrosis (scarring), a cannabinoid found in marijuana, specifically, CBD may contribute to the cell death of hepatic stellate cells that contribute to liver scarring. This suggests that CBD may reduce the extent of scarring in the liver when it is damaged.

Marijuana use in patients with cancer is becoming increasingly common. One of the most commonly reported benefits is the reduction of nausea and vomiting for patients in chemotherapy. Besides reducing the unpleasant symptoms of chemotherapy, marijuana shows potential as a cancer therapy itself. In mice and rat models, researchers have found that THC and other cannabinoids can trigger cell death in many types of cancer cells. In 2007, researchers at Harvard University found that THC may reduce the size of human lung tumors implanted into rats and mice. The reduction in tumor mass and volume were found to be as high as 50%, and the reduction of cancerous lesions in the lungs was around 60%.

Using marijuana may be beneficial for patients living with different forms of inflammatory bowel disease, including Crohn's disease and ulcerative colitis. Observational studies indicate that using marijuana can result in an improvement in symptoms, as well as a reduction in the use of standard medications. In a 2014 study, researchers found that IBD patients who used marijuana showed greater improvements in symptoms than those who received a placebo. The study found that of the 11 participants given marijuana, 5 were able to achieve complete remission of the disease. Inflammation: Tambe Y, Tsujiuchi H, Honda G, et al. 1996. Gastric cytoprotection of the non-steroidal anti-inflammatory sesquiterpene, beta-caryophyllene. Planta Med, 62:469-70.

Geriatric syndromes (GS) are multifactorial, and shared risk factors—including older age, cognitive impairment, functional impairment, and impaired mobility—were demonstrated across the common geriatric syndromes of pressure ulcers, incontinence, falls, functional decline, and delirium. The term “geriatric syndrome” is used to capture those clinical conditions in older persons that do not fit into discrete disease categories. Many of the most common conditions cared for by geriatricians, including delirium, falls, frailty, dizziness, syncope and urinary incontinence, are classified as geriatric syndromes.

Cannabis and cannabinoids effects are useful for geriatric syndromes patients while the side effects of cannabis are trivial in comparison to drug products medication. Cannabinoids are alleviating pain, specifically skeletal and neuropathic pains, are bone builders, increasing appetite and are sedatives. However not all the cannabinoids and cannabis extracts are the same and practically many cannabis extracts are having opposite effects. Some are sedatives, and some are alerting. No single cannabinoid is as effective as the mixture of cannabinoids, termed “entourage effect”.

Cannabis may increase the risk of falls, increase the risk of confusion, especially in elderly, and therefore it is highly needed to adjust the cannabis composition to match the risks. Recreational legalization will make more seniors open to medical cannabis which is not standardized and typically tends to have high THC levels which increases the risk of falls and confusion by GSs patients and elderly population.

Direct association between frailty and elevated circulating levels of interleukin (IL)-6, a proinflammatory cytokine, was first observed in community-dwelling older adults. A large number of studies in many cohorts of older adults and under various care settings have since provided evidence supporting the role of chronic inflammation and immune activation in the pathogenesis of the frailty syndrome.

Chronic pain is the most common reason that physicians recommend marijuana in the states where it is allowed. Marijuana relieves pain without over sedation. Marijuana allows pain patients to use fewer narcotics and fewer nonprescription, or over-the-counter, pain medications.

Marijuana is far safer than anti-depressant medications, tranquilizers and alcohol—alternatives the elderly otherwise seek out to deal with these issues. Marijuana is also an appetite stimulant—it causes “the munchies.” This can help an aging population to maintain an appropriate weight and fight off cachexia, or the wasting syndrome. The frail elderly can be turned into a more robust elderly with marijuana or cannabinoids therapy.

The present invention relates to a composition of matter of cannabis composition to treat elderly population and geriatric syndromes patients, with precise and highly beneficial cannabis compositions to treat and alleviate the complex conditions and plethora of the geriatric syndromes manifestations and improve quality of life.

Cannabis is used to treat various diseases that are very common in elderly population, however, currently there is no accepted cannabis standardization, cannabis mode of administration that is specific and convenient for the elderly GS patients. Elderly are using cannabis by smoking, vaping, edible cookies and the like.

The current invention is useful in improving elderly and GS patients's quality of life, reducing use of drugs, the poly-pharmacy, improving wellbeing, improving insomnia, improving alertness and personal activity, reducing intensity of inflammation, improving cognitive state.

There is an unmet need to provide a cannabis anti-inflammatory and pain control composition that will provide the cannabis effects without reducing the elderly patient alertness during the day time and a different and matching cannabis composition for the night time that will be relaxing, anti-depressant and provide good sleep and alleviate insomnia. Cannabis is effective in alleviating the geriatric syndrome by improving sleep, increasing appetite, reducing spasticity and pain, anti-inflammation, reduce depression, improve osteoporosis, reduce polypharmacy the use of too many drugs with tremendous side effects reducing quality of life and numerous identified and unidentified contra indications, and alleviating delirium and frailty.

In certain embodiments, there is provided a solid self-emulsifying cannabinoid composition, comprising:
a. from about 0.1% to about 30% by weight of at least one cannabinoid or a mixture thereof, and
b. from about 1.0% to about 5% by weight of at least one essential oil or at least one terpene or a mixture thereof, and
c. from about 1% to about to about 40% of at least two emulsifiers, and
d. from about 40% to about 90% of at least one adsorbing powder,
wherein the at least one cannabinoid is essentially solubilized in the mixture of the at least one terpene, at least one essential oil and at least two emulsifiers,
wherein the resulting mixture is adsorbed onto the at least one adsorbing powder affording a solid self-emulsifying composition, and
wherein said solid composition self emulsifies upon contact with water or mammal's body fluids to produce an oil-in-water sub-micron emulsion comprising a plurality of particles having a mean particle size of from about 10 nm to about 1,000 nm.

In some embodiments, the composition of this invention comprise at least one cannabinoid selected from the group consisting of a natural or synthetic cannabinoid, cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and combinations thereof in a concentration of from about 0.01% w/w to about 10% w/w or, from about 0.2% w/w to about 5% w/w.

In some other embodiments, the composition of this invention comprise at least least two emulsifiers, wherein at least one is selected from non-ionic surfactants with HLB of about 10 to about 16 and the other(s) emulsifier is selected from non-ionic hydrophilic or hydrophobic surfactants having a HLB value of about 2 to about 12.

According to some embodiments, the compositions of this invention comprise at least one hydrophilic emulsifier having HLB from about 10 to about 16 is selected from the group consisting of sucrose ester, sucrose stearate, sucrose laurate, sucrose oleate, polysorbate, polyoxyl hydrogenated castor oil, polyoxyl castor oil, tocopherol polyethylene glycol 1000 succinate, polyglyceryl fatty acid ester and combinations thereof and mixtures thereof, in a concentration of from about 1% w/w to about 20% w/w, preferably from about 2% w/w to about 10% w/w.

According to some other embodiments, the compositions of this invention comprise at least one emulsifier having HLB from about 2 to about 12 selected from the group consisting of sorbitan fatty acid esters, sorbitan monooleate, sorbitan stearate, sorbitan oleate, sucrose ester, sucrose di and tri stearate, polyglyceryl fatty acid esters, polyglyceryl-3 dioleate, fatty acids esters and ethers, steareat-2, oleth-2, ceteth-2, salts thereof, derivatives thereof, and combinations thereof.

According to some embodiments, the compositions of this invention comprise at least one terpene selected from bisabolol, borneol, caryophyllene, carene, camphene, cineol, citronella, eucalyptol, geraniol, guaiol, humulene, isopropyltoluene, isopulegol, linalool, limonene, methyl salicylate, menthol, myrcene, nerolidol, ocimene, pinene, phytol, pulegone, terpinene, terpinolene, thymol, salts thereof, derivatives thereof and mixtures thereof.

In some embodiments, the compostions of this invention comprise at least one essential oil selected from a Cannabis species, Allspice Berry, Amber Essence, Anise Seed, Mica, Balsam of Peru, Basil, Bay Leaf, Benzoin Gum, Bergamot, Bois de Rose (Rosewood), Cajuput, Calendula (Marigold pot), White Camphor, Caraway Seed, Cardamone, Carrot Seed, Cedarwood, Celery, German or Hungarian Chamomile, Roman or English Chamomile, Cinnamon, Citronella, Clary Sage, Clovebud, Coriander, Cumin, Cypress, Eucalyptus, Fennel, Siberian Fir needle, Frankincense (Olibanum oil), Garlic, Rose Geranium, Ginger, Grapefruit, Hyssop, Jasmine Absolute, Jojoba, Juniper Berry, Lavender, Lemon, Lemongrass, Lime, Sweet Marjoram, Mugwort, Mullein Flower, Myrrh Gum, Bigarade Neroli, Nutmeg, Bitter Orange, Sweet Orange, Oregano, Patchouly, Pennyroyal, Black Pepper, Peppermint, Petitegrain, Pine Needle, Poke Root, Rose Absolute, Rosehip Seed, Rosemary, Dalmatian Sage, Sandalwood Oil, Sassafras, Spearmint, Spikenard, Spruce (Hemlock), Tangerine, Tea Tree, Thuja (Cedar leaf), Thyme, Vanilla extract, Vetivert, Wintergreen, an extract selected from Witch Hazel (Hamamelia) Extract, and Ylang Ylang (Cananga) Extract and combinations thereof.

According to some other embodiments, the compositions of this invention comprise at least one adsorbing powder selected from the group consisting of silicon dioxide, colloidal silica, fumed silica (Aerosil®), Magnesium Aluminum Silicate (Neusilin®), di- and tribasic calcium phosphates, calcium carbonate, calcium silicate and combinations thereof, in a concentration of from about 20% w/w to about 90% w/w, from about 40% to about 80%, or from about 50% w/w to about 70% w/w.

In some embodiments, the compositions of this invention comprise at least 90%, at least 95%, at least 98% or at least 99% of the particle plurality in the self-emulsified emulsion below about 1,000 nm, below about 800 nm, below about 600 nm, below about 400 nm or below about 200 nm.

In some other embodiments, the composition of this invention have a ratio of the at least one cannabinoid to the at least two emulsifiers from about 5:1 to about 1:20, from about 2:1 to about 1:10, or from about 1:1 to about 1:5 on weight basis.

According to some embodiments, the compositions of this invention are formulated in a dosage form selected from a tablet, a granule, a capsule, a lozenge, a hard shell capsule, a soft shell capsule, a powder for reconstitution, granules to be administered by a spoon, by a volume measuring device or mixed with nutrients or food a powder, granules, pellets or micro-particles packed in a sachet, a unit dose package, a suspension in a liquid or in a syrup, a candy, a toffee, a chocolate a cookie or an enema.

According to some other embodiments, the compositions of this invention are formulated as oral or mucosal dosage forms, dissolving in mouth or muco-adhesive, enteric coated, and formulated as immediate release, slow or controlled release.

According to some embodiments, the compositions of this invention comprise cannabidiol (CBD) as the at least one cannabinoid

In certain embodiments, there is provided a method of treating cannabinoid treatable disorders, by administering to a mammal subject a therapeutically effective amount of the composition of this invention, wherein the composition exhibits an improved oral cannabinoid bioavailability, higher by at least about 50% than the bioavailability of the same at least one cannabinoid when dissolved in an organic solvent or mixture of organic solvents.

In certain embodiments, there is provided a method of treatment for inducing an alerting response in a mammal subject in need thereof by administration of a therapeutically effective amount of the composition of this invention, wherein comprising at least one cannabinoid selected from at least about 1 mg of cannabidiol (CBD), a derivative thereof and combinations thereof and at least about 1 mg of at least one alerting terpene per one serving, wherein the at least one alerting terpene is selected from limonene, alfa and beta pinene, orange terpenes, thymol, isoborneol, isoeugenol and combinations thereof, or citronella or orange essential oils or lavender essential oil, caryophyllene, rosmarinus oil, citrus oil and combinations thereof.

In certain embodiments, there is provided a method of treatment for sedation and/or sleep inducing in a mammal in need thereof, by administration at about one hour before retiring to sleep or as needed of a therapeutically effective amount of the above composition of this invention, wherein comprising from about 1 mg to about 10 mg of at least one sedating cannabinoid and from about 0.5 mg to about 5 mg of at least one sedating terpene per unit serving.

In some embodiments, there is provided the above method of treatment, wherein the sedating cannabinoid is selected from Tetrahudrocannabinol (THC), Cannabinol (CBN), Cannabidiol and combinations thereof, and the sedating terpene is selected from myrcene, linalool, linalyl acetate, alfa terpineol and citronellal, sandalwood, lavender, valerian, neroli essential oils and combinations thereof.

In some other embodiments, there is provided a kit for the treatment of geriatric syndroms and improvement of elderly wellbeing and treatment of neurological patients, epilepsy, PTSD, anxiety, schizophrenia, Parkinson's disease or auto-immune diseases, which are also suffering from insomnia and are in need of improvement of their wellbeing comprising a night composition of this invention, comprising:
a) at least about 1 mg to about 20 mg of cannabis extract or at least one cannabinoid or derivatives thereof and
b) at least about 1 mg of at least one sedative terpene.
and a day time composition comprising
a) at least about 10 mg cannabis extract or at least one cannabinoid or derivatives thereof having a CBD:THC ratio of about 10 to about 200 and
b) at least about 1 mg of at least one alerting terpene.

According to the principles of the present invention, and without being limited to any theory or mechanism, in certain embodiments, the at least one annabinoid, terpene, essential oil, emulsifier and sorbent in each formulation can be substituted with another sannabinoid, oil, terpene, emulsifier and sorbent in the same or substantially the same % by weight. In certain embodiments, the pharmaceutical composition comprises or consists of a formulation having a cannabinoid/terpene or oil/emulsifier/sorbent ratio as a formulation in the Tables of the examples. Each possibility represents a separate embodiment of the invention.

### EXAMPLES

### Example 1

TABLE 1
Solid and self-emulsifying cannabinoids compositions of pure THC and high
THC extract

| Formula # | 1A | 1B | 1C | 1D | 1E | 1F | 1G |
|---|---|---|---|---|---|---|---|
| Ingredient | % W/W | % W/W | % W/W | % W/W | % W/W | % W/W | % W/W |
| Afghan indica pure extract | 1 | 2 | 3 | 1 | 2 | 3 | 0 |
| THC | 0 | 0 | 0 | 0 | 0 | 0 | 1 |
| Capric/caprylic triglyceride | 4 | 6 | 8 | 4 | 6 | 8 | 10 |
| Sucrose stearate | 0.8 | 1.5 | 2.5 | 0.8 | 1.5 | 2.5 | 2.5 |
| Tocopheryl PEG 1000 succinate | 0.8 | 1.5 | 2.5 | 0.8 | 1.5 | 2.5 | 2.5 |
| PEG-35 Castor Oil | 0.8 | 1.5 | 2.5 | 0.8 | 1.5 | 2.5 | 2.5 |
| Micro crystalline cellulose | 60 | 50 | 40 | 60 | 50 | 40 | 40 |
| Fumed silica | 32.6 | 37.5 | 41.5 | 32.6 | 37.5 | 41.5 | 41.5 |
| Total | 100 | 100 | 100 | 100 | 100 | 100 | 100 |
| Ratio of oil to absorbent | 0.08 | 0.14 | 0.23 | 0.08 | 0.14 | 0.23 | 0.23 |

### Example 2

TABLE 2
Solid and self-emulsifying cannabinoids compositions of cannabidiol (CBD)
and high CBD:THC ration extract

| Formula # | 2A | 2B | 2C | 2D | 2E | 2F | 2G |
|---|---|---|---|---|---|---|---|
| Ingredient | % W/W | % W/W | % W/W | % W/W | % W/W | % W/W | % W/W |
| Cannabidiol | 2 | 4 | 8 | 10 | 0 | 0 | 0 |
| ACDC ™ strain pure oil extract | 0 | 0 | 0 | 0 | 10 | 20 | 20 |
| Capric/caprylic triglyceride | 10 | 20 | 30 | 20 | 20 | 20 | 20 |
| Sucrose stearate | 2 | 5 | 0 | 0 | 4 | 6 | 0 |
| Tocopheryl PEG 1000 succinate | 2 | 5 | 6 | 6 | 4 | 6 | 6 |
| PEG-35 Castor Oil | 2 | 5 | 6 | 6 | 4 | 6 | 6 |
| Sorbitan laurate | 0 | 0 | 6 | 6 | 0 | 0 | 6 |
| Micro crystalline cellulose | 60 | 50 | 40 | 50 | 50 | 40 | 40 |
| Fumed silica | 22 | 11 | 4 | 2 | 8 | 2 | 2 |
| Total | 100 | 100 | 100 | 100 | 100 | 100 | 100 |
| Ratio of oil to absorbent | 0.22 | 0.64 | 1.14 | 0.81 | 0.72 | 1.38 | 1.24 |

### Example 3

TABLE 3
Solid and self-emulsifying cannabinoids compositions of cannabidiol (CBD)
and high CBD:THC ratio extract absorbed on magnesium alumino-metasilicate
(Neusilin ®)

| Formula # | 3A | 3B | 3C | 3D | 3E | 3F | 3G |
|---|---|---|---|---|---|---|---|
| Ingredient | % W/W | % W/W | % W/W | % W/W | % W/W | % W/W | % W/W |
| Cannabidiol | 4 | 4 | 4 | 6 | 0 | 0 | 0 |
| Charlotte web ™ oil pure extract | 0 | 0 | 0 | 0 | 10 | 20 | 30 |
| Capric/caprylic triglyceride | 20 | 20 | 20 | 30 | 20 | 20 | 10 |
| Sucrose stearate | 6 | 5 | 0 | 0 | 0 | 0 | 0 |
| Tocopheryl PEG 1000 succinate | 6 | 5 | 4 | 6 | 5 | 5 | 6 |
| PEG-35 Castor Oil | 6 | 5 | 4 | 6 | 5 | 5 | 6 |
| Sorbitan laurate | 6 | 0 | 6 | 6 | 5 | 5 | 6 |
| Magnesium Alumino-metasilicate. | 44 | 57 | 55 | 30 | 50 | 40 | 30 |
| Micro crystalline cellulose | 0 | 0 | 0 | 4 | 0 | 5 | 10 |
| Fumed silica | 8 | 4 | 7 | 12 | 5 | 0 | 2 |
| Total | 100 | 100 | 100 | 100 | 100 | 100 | 100 |
| Ratio of oil phase to absorbent | 0.81 | 0.64 | 0.52 | 1.04 | 0.73 | 1.11 | 1.24 |

The cannabis pure extract in Table 1 and 2 and 3 were produced by an ethanol extraction of decarboxylated plant material, and comprises about 80% by weight of THC and CBD, and about 2% by weight terpenes.

For determining the “maximal particle size” or the “Largest detected size”, the tested formulation is mixed 1:10 with distilled water at room temperature and slightly rotated until emulsification occur. A drop is then placed on bearing glass and covered with top glass, and the sample is immediately examined with light microscope magnification ×200 or ×400 with calibrated scale.

The solid composition was prepared by combining the cannabinoids with the oils and the emulsifiers and heating to about 70° C. to about 80° C. and vigorously mixing until homogeneity obtained, the mixture of the adsorbing powder was added in portion under mixing without further heating, until a full adsorption was obtained. The amount of the adsorbing powder is adjusted in order to obtain desired properties of the solid dispersion.

All emulsified cannabinoid composition of table 1 and in table 2 and 3 where tested with light microscope for particle size and had mean visible particle size below 800 nm. Accurate mean particle size cannot be examined in light microscope since the particle size is below the visible wavelength and using the Malvern™ nanosizer is not possible since the adsorbing powders are insoluble and avoid detection of the diffracting laser beam and obtaining such data.

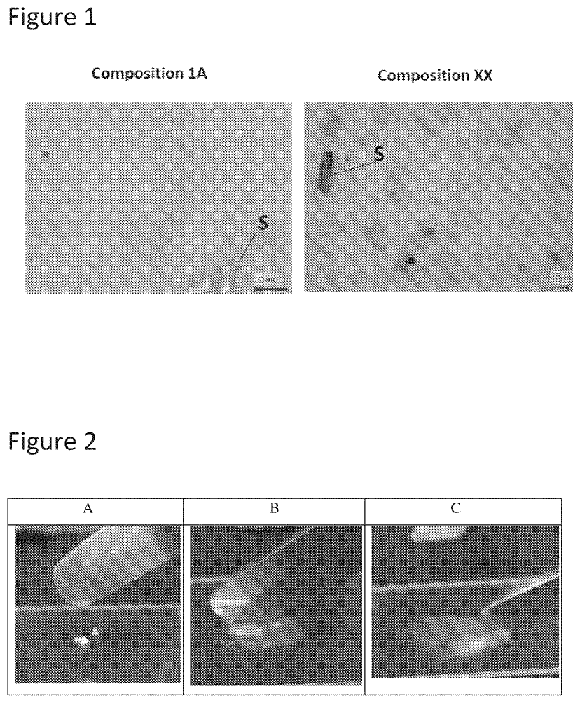

In FIG. 1, a light microscope picture of composition 1A (Nikon, Eclipse™ E200) magnification ×400 it can be seen that the droplet particles are below one micron and no large “oily” particle over one micron are detected. Some large particles of the adsorbing powder which is not soluble in water and which have typical cubic or crystalline shape (indicated as ‘S’ in the figure) can also be detected. FIG. 2 demonstrates the self-emulsification process, whereas a solid particle, a pellet, is mixed with water solution (simulated intestinal fluid) to produce a disperseed of a) the sorbent and b) a fine oil-in-water sub micron emulsion. Panel A of FIG. 2 shows a pellet with a size of about 3 mm placed on a glass slide, while panel B illustrates the addition of a drop of SIF at room temperature. Finally, the immediate formation of a sub-micron emulsion is shown in panel C.

### Example 4

Compositions of solid and self-emulsifying cannabinoid 4A, was produced in a granules or pellets dosage form. All the ingredients of the “oily phase” were mixed; a) 20.0 gram of pure tetrahydrocannabinol (THC) was mixed with b) 100.0 grams of capric/caprylic triglyceride oil (MCT oil, Mygliol™), and c) 30 grams of sucrose distearate (Surfhope™ 1811), and d) 30 grams of PEG-35 castor oil (Cremophor™ EL35), and e) 30 grams of tocopheryl PEG1000 succinate (Kolliphor™ TPGS). The oily phase mixture was heated shortly to about 80° C. and homogenized until mixture was homogeneous, 300 grams of a mixture of micro crystalline cellulose (MCC, Avicel™ 611) and fumed silica (Aerosil™ 200) in ratio of 2:1 was added in small parts under vigorous agitation, the first part was added and mixed while heating to about 70° C. and for the later parts mixing, the heating was stopped. A solid waxy granular mass was obtained.

Compositions of solid and self-emulsifying cannabinoid 4B, was produced in a granules or pellets dosage form. A mixture of 40.0 gram of high CBD cannabis extract (ACDC™ strain) with CBD:THC ratio of 20:1 was mixed with 100.0 grams of capric/caprylic triglyceride oil (MCT oil, Mygliol™), 30 grams of sucrose distearate (Surfhope™ 1811), 30 grams of PEG-35 castor oil (Cremophor™ EL35), and 30 grams of tocopheryl PEG1000 succinate (Kolliphor™ TPGS). The mixture was heated shortly to about 80° C. and homogenized until mixture was homogeneous, 300 grams of mixture of micro crystalline cellulose (MCC, Avicel™ 611) and fumed silica (Aerosil™ 200) and sodium starch glycolates in ratio of 6:3:1 was added and processed with rotating granulation machine.

### Example 5

Tablets 5A, were produced from the formulation 3A of example 3, with 200 grams of lactose and 100 grams of hydroxypropyl methyl cellulose (Methocel™ K4M) and 30 grams of polyvinyl pyrrolidone (PVP, Plasdone™ K30) and 5 grams of magnesium stearate were added and mixed with high speed planetary mixer. The resulting granular mass was compressed in a tablet press.

TABLE 4
mg of ingredients per one tablet unit dose

| | Composition | |
|---|---|---|
| | 4A | 4B |
| Ingredients | mg/Tablet | mg/Tablet |
| THC | 20 | 0 |
| Charlotte web(TM) extract | 0 | 40 |
| Capric/caprylic triglyceride oil | 100 | 100 |
| Sucrose distearate | 30 | 30 |
| PEG-35 castor oil | 30 | 30 |
| Tocopheryl PEG succinate | 30 | 30 |
| micro crystalline cellulose | 200 | 200 |
| Fumed silica | 100 | 100 |
| Lactose | 200 | 200 |
| polyvinyl pyrolidone | 30 | 0 |
| sodium starch glycolates | | 30 |
| hydroxypropyle methyl cellulose | 100 | 100 |
| magnesium stearate | 5 | 5 |
| Total | 845 | 865 |

### Example 6

Enteric coating of the tablets 5A, and of hard vegetable capsules filled with granules formulation 3A, was produced by coating and drying with a solution of Eudragit™ L100-55 6%, PEG6000 1.2%, Talk 2%, water 6% and isopropyl alcohol (IPA) 84.8%.

Enteric coating of the tablets 4A or 4B, was produced with same coating solution and enteric-coating of granules was produced by spraying the enteric coating solution in a fluidized bed apparatus.

### Example 7

Melt in the mouth tablet 7A is produced from 100 grams of one formulation from 1A to 3F, mixed with 50 grams lactose and 50 grams micro crystalline cellulose (MCC) and 10 grams of Croscarmellose Sodium (Solutab®) and pressed in a tablet press under moderate pressure.

### Example 8

Muco adhesive tablets 8A is produced by mixing 100 grams of one formulation from 1A to 3F with 50 grams of xanthan gum (Xantural™ 3000) and 50 grams of Hydroxypropyl methylcellulose (HPMC) K4M, and 1 gram magnesium stearate with high speed planetary mixer and pressed in a tablet press.

### Example 9. Oral Absorption of CBD in Rats

A tablet of composition of 5A was diluted with water of about 40° C. before administration to the rats to obtain CBD concentration of 12 mg/ml and to obtain the cannabinoid oil-in-water emulsion. The emulsion was administered in gavage to the stomach of Sprague drawly rats weighing 220 gr to 250 gr at 12 mg/kg. Blood sample was obtained at various time points after the oral administration and plasma separated. The CBD concentration in the plasma was tested by LC-MS and the area under the curve calculate. Group one was administered with 12 mg/kg of pure CBD dissolved in “solvent” propylene glycol:ethanol 1:1 solution. Group two received the test formulation.

TABLE 10
Area Under the Curve (AUC) and Cmax of CBD in rats' plasma

| | AUC | Cmax |
|---|---|---|
| Test item | ng/ml/hour | ng/ml |
| Group one: | 90 +/− 12 | 38 |
| 12 mg/kg of CBD dissolved in propylene | | |
| glycol:ethanol | | |
| Group two: | 340 +/− 28 | 124 |
| 12 mg/kg of example 5A | | |

### Example 10

TABLE 11
Solid and self-emulsifying cannabinoids compositions of cannabidiol (CBD)
without triglyceride oil

| Formula # | 5A | 5B | 5C | 5D | 5E | 5F | 5G |
|---|---|---|---|---|---|---|---|
| Ingredient | % W/W | % W/W | % W/W | % W/W | % W/W | % W/W | % W/W |
| Cannabidiol | 10 | 20 | 30 | 30 | 0 | 0 | 0 |
| ACDC oil pure extract | 0 | 0 | 0 | 0 | 10 | 20 | 30 |
| Caryophyllen (or tepens mixture) | 1 | 2 | 3 | 5 | 0 | 0 | 0 |
| Sucrose stearate | 2 | 0 | 6 | 0 | 4 | 6 | 0 |
| Tocopheryl PEG 1000 succinate | 4 | 8 | 12 | 12 | 4 | 6 | 12 |
| PEG-35 Castor Oil | 0 | 8 | 0 | 6 | 4 | 6 | 6 |
| Polyglycery1-3 dioleate | 1 | 4 | 6 | 6 | 0 | 0 | 6 |
| Micro crystalline cellulose | 60 | 50 | 40 | 40 | 50 | 40 | 40 |
| Fumed silica | 22 | 8 | 3 | 1 | 28 | 22 | 6 |
| Total | 100 | 100 | 100 | 100 | 100 | 100 | 100 |
| Ratio of ingredients to absorbent | 0.21 | 0.66 | 1.19 | 1.29 | 0.28 | 0.61 | 1.04 |

### Example 11

TABLE 12
Solid and self-emulsifying cannabinoids compositions of cannabidiol (CBD)
without triglyceride oil

| Formula # | 6A | 6B | 6C | 6D | 6E | 6F | 6G |
|---|---|---|---|---|---|---|---|
| Ingredient | % W/W | % W/W | % W/W | % W/W | % W/W | % W/W | % W/W |
| Cannabidiol | 30 | 30 | 30 | 30 | 0 | 0 | 0 |
| ACDC oil pure extract | 0 | 0 | 0 | 0 | 30 | 30 | 30 |
| Caryophyllen (or tepens mixture) | 2 | 2 | 3 | 5 | 0 | 0 | 0 |
| Sucrose stearate | 4 | 5 | 0 | 0 | 0 | 5 | 0 |
| Tocopheryl PEG 1000 succinate | 6 | 15 | 8 | 15 | 5 | 10 | 10 |
| PEG-35 Castor Oil | 4 | 5 | 6 | 5 | 5 | 0 | 6 |
| Sorbitan laurate | 4 | 0 | 0 | 0 | 5 | 0 | 0 |
| Polyglycery1-3 dioleate | 0 | 0 | 6 | 5 | 0 | 6 | 6 |
| Magnesium Alumino-metasilicate. | 44 | 40 | 45 | 30 | 50 | 40 | 30 |
| Micro crystalline cellulose | 0 | 0 | 0 | 5 | 0 | 5 | 10 |
| Fumed silica | 6 | 3 | 2 | 5 | 5 | 4 | 8 |
| Total | 100 | 100 | 100 | 100 | 100 | 100 | 100 |
| Ratio of ingredients to absorbent | 0.92 | 1.33 | 1.00 | 1.38 | 0.73 | 0.92 | 0.96 |

The solid composition was prepared by combining the cannabinoids with the terpenes and the emulsifiers and heating to about 70° C. to about 80° C. and vigorously mixing until homogeneity obtained, the mixture of the adsorbing powder was added in portion under mixing without further heating, until a full adsorption was obtained. The amount of the adsorbing powder is adjusted in order to obtain desired properties of the solid dispersion.

All emulsified cannabinoid composition of table 11 and in table 12 where tested with light microscope for particle size and had visible mean particle size below 1,000 nm. Accurate mean particle size cannot be examined in light microscope since the particle size is below the visible wavelength and using the Malvern™ nanosizer is not possible since the adsorbing powders are insoluble and avoid detection of the diffracting laser beam and obtaining such data.

1. A solid self-emulsifying cannabinoid composition, comprising:
a. from about 0.1% to about 30% by weight of at least one cannabinoid or a mixture thereof, and
b. from about 1.0% to about 5% by weight of at least one essential oil or at least one terpene or a mixture thereof, and
c. from about 1% to about to about 40% of at least two emulsifiers, and
d. from about 40% to about 90% of at least one adsorbing powder,
wherein the at least one cannabinoid is essentially solubilized in the mixture of the at least one terpene, at least one essential oil and at least two emulsifiers,
wherein the resulting mixture is adsorbed onto the at least one adsorbing powder affording a solid self-emulsifying composition, and
wherein said solid composition self emulsifies upon contact with water or mammal's body fluids to produce an oil-in-water sub-micron emulsion comprising a plurality of particles having a mean particle size of from about 10 nm to about 1,000 nm.

2. The composition of claim 1, wherein the at least one cannabinoid is selected from the group consisting of a natural or synthetic cannabinoid, cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and combinations thereof in a concentration of from about 0.01% w/w to about 10% w/w or, from about 0.2% w/w to about 5% w/w.

3. The composition of claim 1, wherein at least one of the at least two emulsifiers is selected from non-ionic surfactants with HLB of about 10 to about 16 and the other(s)
emulsifier is selected from non-ionic hydrophilic or hydrophobic surfactants having a HLB value of about 2 to about 12.

4. The composition of claim 3, wherein the at least one hydrophilic emulsifier having HLB from about 10 to about 16 is selected from the group consisting of sucrose ester, sucrose stearate, sucrose laurate, sucrose oleate, polysorbate, polyoxyl hydrogenated castor oil, polyoxyl castor oil, tocopherol polyethylene glycol 1000 succinate, poly glyceryl fatty acid ester and combinations thereof and mixtures thereof, in a concentration of from about 1% w/w to about 20% w/w, preferably from about 2% w/w to about 10% w/w.

5. The pharmaceutical composition of claim 3, wherein the at least one emulsifier having HLB from about 2 to about 12 is selected from the group consisting of sorbitan fatty acid esters, sorbitan monooleate, sorbitan stearate, sorbitan oleate, sucrose ester, sucrose di and tri stearate, polyglyceryl fatty acid esters, polyglyceryl-3 dioleate, fatty acids esters and ethers, steareat-2, oleth-2, ceteth-2, salts thereof, derivatives thereof, and combinations thereof.

6. The composition of claim 1, wherein the at least one terpene is selected from bisabolol, borneol, caryophyllene, carene, camphene, cineol, citronella, eucalyptol, geraniol, guaiol, humulene, isopropyltoluene, isopulegol, linalool, limonene, methyl salicylate, menthol, myrcene, nerolidol, ocimene, pinene, phytol, pulegone, terpinene, terpinolene, thymol, salts thereof, derivatives thereof and mixtures thereof.

7. The pharmaceutical composition of claim 1, wherein the at least one adsorbing powder is selected from the group consisting of silicon dioxide, colloidal silica, fumed silica (Aerosil®), Magnesium Aluminum Silicate (Neusilin®), di- and tribasic calcium phosphates, calcium carbonate, calcium silicate and combinations thereof, in a concentration of from about 20% w/w to about 90% w/w, from about 40% to about 80%, or from about 50% w/w to about 70% w/w.

8. The composition of claim 1, wherein at least 90% at least 95%, at least 98% or at least 99% of the particle plurality in the self-emulsified emulsion are below about 1,000 nm, below about 800 nm, below about 600 nm, below about 400 nm or below about 200 nm.

9. The composition of claim 1, wherein the ratio of the at least one cannabinoid or cannabinoids to the at least two emulsifiers is from about 5:1 to about 1:20, from about 2:1 to about 1:10, or from about 1:1 to about 1:5 on weight basis.

10. The composition of claim 1, wherein formulated in a dosage form selected from a tablet, a granule, a capsule, a lozenge, a hard shell capsule, a soft shell capsule, a powder for reconstitution, granules to be administered by a spoon, by a volume measuring device or mixed with nutrients or food a powder, granules, pellets or micro particles packed in a sachet or a unit dose package or suspended in a liquid or in a syrup, a candy, a toffee, a chocolate or cookie or an enema.

11. The composition of claim 1, wherein the dosage form is oral or mucosal, dissolving in mouth or muco-adhesive, enteric coated, formulated as immediate release, slow or controlled release.

12. The composition of claim 1, wherein the at least one cannabinoid is cannabidiol (CBD).

13. A method of treating cannabinoid treatable disorders, by administering to a mammal subject a therapeutically effective amount of the composition of any of the claims above, wherein the composition exhibits an improved oral cannabinoid bioavailability, higher by at least about 50% than the bioavailability of the same at least one cannabinoid when dissolved in an organic solvent or mixture of organic solvents.

14. A method of treatment for inducing an alerting response in a mammal subject in need thereof by administration of a therapeutically effective amount of the composition of claim 1, wherein comprising at least one cannabinoid selected from at least about 1 mg of cannabidiol, a derivative thereof and combinations thereof and at least about 1 mg of at least one alerting terpene per one serving, wherein the at least one alerting terpene is selected from limonene, alfa and beta pinene, orange terpenes, thymol, isoborneol, isoeugenol and combinations thereof, or citronella or orange essential oils or lavender essential oil, caryophyllene, rosmarinus oil, citrus oil and combinations thereof.

15. A method of treatment for sedation and/or sleep inducing in a mammal in need thereof, by administration at about one hour before retiring to sleep or as needed of a therapeutically effective amount of the composition of claim 1, wherein comprising from about 1 mg to about 10 mg of at least one sedating cannabinoid and from about 0.5 mg to about 5 mg of at least one sedating terpene per unit serving.

16. The method of claim 16, wherein the sedating cannabinoid is selected from Tetrahudrocannabinol (THC), Cannabinol (CBN), Cannabidiol (CBD) and combinations thereof, and the sedating terpene is selected from myrcene, linalool, linalyl acetate, alfa terpineol and citronellal, sandalwood, lavender, valerian, neroli essential oils and combinations thereof.

17. A kit for the treatment of geriatric syndroms and improvement of elderly wellbeing and for neurological patients, epilepsy, PTSD, anxiety, schizophrenia, Parkinson's disease or auto-immune diseases, which are also suffering from insomnia and are in need of improvement of their wellbeing comprising a night composition according to claim 1, comprising:
a) at least about 1 mg to about 20 mg of cannabis extract or at least one cannabinoid or derivatives thereof and
b) at least about 1 mg of at least one sedative terpene.
and a day time composition comprising
a) at least about 10 mg cannabis extract or at least one cannabinoid or derivatives thereof having a CBD:THC ratio of about 10 to about 200 and b) at least about 1 mg of at least one alerting terpene.
